(19) United States
(12) Patent Application Publication
(10) Pub. No.: US 2022/0117328 A1
(43) Pub. Date: Apr. 21, 2022

(54) ADJUSTABLE FACE SHIELD

(71) Applicant: KodifyMe FreeSpacers, Inc., Chester, PA (US)

(72) Inventor: Josie LAMKIN, Philadelphia, PA (US)

(21) Appl. No.: 17/483,035

(22) Filed: Sep. 23, 2021

Related U.S. Application Data
(60) Provisional application No. 63/094,628, filed on Oct. 21, 2020.

(51) Int. Cl. A41D 13/11

(52) U.S. Cl. CPC A41D 13/11; A61F 9/06

(57) ABSTRACT

A multi-adjustment face shield is disclosed having a flexible front plate or shield, a foam flexible forehead support member, and an adjustable, stretchable headband. The top shield is positioned to be directly in front of a person's eyes wearing the face shield. The bottom shield is removable. The face shield is surrounded by an adequate, peripheral margin, frame, or window surrounding portion, which provides an anti-glare characteristic or capability to the margin. The flexible forehead support member is provided on the upper part of the transparent member to nestle against a user's forehead.

### CROSS-REFERENCE TO RELATED APPLICATION

This application claims priority from U.S. Provisional Patent Application No. 63/094,628, titled “Adjustable Face Shield”, filed on Oct. 21, 2020, the entire contents of which are incorporated by reference herein.

### BACKGROUND OF THE DISCLOSURE

### 1. Field of the Disclosure

The present disclosure presents a novel mobile design in the arena of face shields for protective purposes. It comes with multi-adjustable options, as the name suggests. The possibilities and flexibility in the present disclosure provide the Adjustable Face Shield application toward usages such as in various industries, professions, and hobbies, e.g., medicine, dentistry, painting, manufacturing, construction site, welding, and the like. The Adjustable Face

Shield is easy-to-use and somewhat ambidextrous, can be fitted to every face and head because of flexible mounts, mounting bands, and slits. All in all, the design is novel and a regime-changer in the arena of face-shields.

### 2. Background

It has long been advantageous for individuals working with liquids to wear a protective face shield to prevent those liquids from splattering or spattering on the face. For example, face protection in medical procedures to avoid the splattering of blood on the face is recommended. It is also advantageous in medical procedures to use multi-adjustment surgical equipment where possible to substantially reduce the risk of infection and the cost of sterilizing the surgical equipment. Additionally, cleaning face shields of deposited chemicals, such as blood, paint, adhesives, dyes, solvents, resins, and the like, is time-consuming and often ineffective. Thus, multi-adjustment face shields have seen a complete application in various industries, e.g., medicine, dentistry, painting, manufacturing, and the like. Because of the complete application and use of multi-adjustment face shields, the mounting band or bands which secure the shield to the face must be adjustable to accommodate a variety of different sized faces and heads. Some prior art face shields utilize a fastener or integral member to attach the band to the shield. An over the headband having a width greater than the width of the slits is looped through the slits. The bands are threaded in a particular direction through the slits. Pulling on the ends of the band, so threaded, decreases the amount of band positioned to hold the shield to a user's head, thus making a secure fit. However, such slits, and therefore multi-adjustment face shields having those slits, can be improved upon as will be disclosed in the present application.

### BRIEF SUMMARY OF THE DISCLOSURE

A multi-adjustment face shield having an improved attachment mechanism for securing a band to attach the shield to a user's face is disclosed. The multi-adjustment face shield includes a flexible transparent member having an overhead band therethrough. The overhead band has points that grab at selected locations to adjust the face shield to the user's head. A flexible forehead support member is provided on the upper portion of the transparent member to nestle against a user's forehead. The face shield has a flexible transparent portion with a flexible forehead support member and is held on a user's head by a headband. The headband is threaded through one slit in the suitable flexible transparent portion. The edges of the slits have points that correspond to the headband and keep the band at the desired position firmly in the slits. This is how the face shield ensures the desired tightness while remaining in position on the user. The pointed teeth or edges of the slit press into the surfaces of the band. In another version, face shields having slits are available without a band attached.

Briefly stated, one aspect of the present disclosure is directed to a face shield having a flexible, upper transparent member having a flexible headband extending from one side of the upper transparent member to an opposing side of the upper transparent member and dimensioned to extend around a portion of a head of a user. The flexible headband is configured to removably secure the upper transparent member to the head of the user. A flexible cushion member is disposed on a posterior side of the upper transparent member, the flexible cushion member being configured to provide a cushion barrier between the upper transparent member and a forehead of the user. A flexible, bottom member is removably attached to the upper transparent member, a first side of the bottom member having a first elastic band attached thereto and a second side of the bottom member having a second elastic band attached thereto, each elastic band having both ends thereof attached to the respective side of the bottom member, whereby the first and second elastic bands are each configured to loop around a respective ear of the user to removably secure the bottom member to the head of the user.

In one configuration, the face shield further includes an adhesive strip positioned along an anterior side of the upper transparent member, the adhesive strip being configured to removably receive a decorative component thereon. In one such configuration, the adhesive strip is a double-sided adhesive strip.

In any one of the previous configurations, the flexible cushion member is constructed of memory foam. In one such configuration, the flexible cushion member is constructed of bamboo memory foam.

In any one of the previous configurations, the upper transparent member defines a thickness between approximately 0.20 mm and approximately 0.24 mm.

In any one of the previous configurations, at least one end of the headband is removably attached to the upper transparent member.

In any one of the previous configurations, the bottom member is also a transparent member.

In any one of the previous configurations, the upper transparent member and the bottom member are removably attached via hook-and-loop fastener strips.

In any one of the previous configurations, a posterior side of the bottom member is removably attached to an anterior side of the upper transparent member. Briefly stated, one aspect of the present disclosure is directed to a face shield having a flexible, upper transparent member having a flexible headband extending from one side of the upper transparent member to an opposing side of the upper transparent member and dimensioned to extend around a portion of a head of a user. The flexible headband is configured to removably secure the upper transparent member to the head of the user, wherein at least one end of the headband is removably attached to the upper transparent member. A memory foam, flexible cushion member is disposed on a posterior side of the upper transparent member, the flexible cushion member being configured to provide a cushion barrier between the upper transparent member and a forehead of the user. An adhesive strip is positioned along an anterior side of the upper transparent member, the adhesive strip being configured to removably receive a decorative component thereon. A flexible, bottom transparent member is removably attached to the upper transparent member, a first side of the bottom transparent member having a first elastic band attached thereto and a second side of the bottom transparent member having a second elastic band attached thereto, each elastic band having both ends thereof attached to the respective side of the bottom transparent member, whereby the first and second elastic bands are each configured to loop around a respective ear of the user to removably secure the bottom transparent member to the head of the user. A posterior side of the bottom transparent member is removably attached to the anterior side of the upper transparent member via hook-and-loop fastener strips.

In one configuration, the adhesive strip is a double-sided adhesive strip.

### BRIEF DESCRIPTION OF THE SEVERAL VIEWS OF THE DRAWINGS

The following description of the disclosure will be better understood when read in conjunction with the appended drawings. It should be understood, however, that the disclosure is not limited to the precise arrangements and instrumentalities shown. In the drawings:

FIG. 1 is a top and anterior, perspective view of a face shield according to the present disclosure;

FIG. 2 is an anterior perspective view of the face shield of FIG. 1, with an upper transparent member thereof separated from a bottom member thereof;

FIG. 3 is a posterior perspective view of the face shield of FIG. 1, with the upper transparent member thereof separated from the bottom member thereof;

FIG. 4 is an anterior elevational view of the face shield of FIG. 1;

FIG. 5 is a posterior elevational view of the face shield of FIG. 1;

FIG. 6 is a dorsal plan view of the face shield of FIG. 1;

FIG. 7 is a ventral plan view of the face shield of FIG. 1;

FIG. 8 is a right-side elevational view of the face shield of FIG. 1;

FIG. 9 is a left-side elevational view of the face shield of FIG. 1;

FIG. 10A is an anterior elevational view of the face shield of FIG. 1 worn by a user;

FIG. 10B is a left-side elevational view of the face shield of FIG. 1 worn by a user; and

FIGS. 11-14C illustrate the face shield of FIG. 1 with various exemplary decorative components attached thereto.

### DETAILED DESCRIPTION OF THE DISCLOSURE

Certain terminology is used in the following description for convenience only and is not limiting. The words “lower,” “bottom,” “upper” and “top” designate directions in the drawings to which reference is made. The words “inwardly,” “outwardly,” “upwardly” and “downwardly” refer to directions toward and away from, respectively, the geometric center of the face shield, and designated parts thereof, in accordance with the present disclosure. In describing the face shield , the term proximal is used in relation to the upper end of the device and the term distal is used in relation to the bottom surface of the device. Unless specifically set forth herein, the terms “a,” “an” and “the” are not limited to one element, but instead should be read as meaning “at least one.”

As used herein, the term “and/or” includes any and all combinations of one or more of the associated listed items. It will be further understood that the terms “comprises” and/or “comprising,” when used in this specification, specify the presence of stated features, steps, operations, elements, and/or components, but do not preclude the presence or addition on one or more other features, steps, operations, elements, components, and/or groups thereof. The terminology includes the words noted above, derivatives thereof and words of similar import. The terminology used herein is to describe particular embodiments only and is not intended to be limiting of the present disclosure.

Unless otherwise defined, all terms (including technical and scientific terms) used herein have the same meaning as commonly understood by one having ordinary skill in the topic of the face shield. It will be further understood that terms, such as those defined in commonly-used dictionaries, should be interpreted as having that is consistent with their meaning in the context of protective gears related to face, and its dependency on necessary accessories; and the present disclosure and will not be interpreted in an idealized and overly formal sense unless expressly so defined herein.

It should also be understood that the terms “about,” “approximately,” “generally,” “substantially” and like terms, used herein when referring to a dimension or characteristic of a component of the disclosure, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

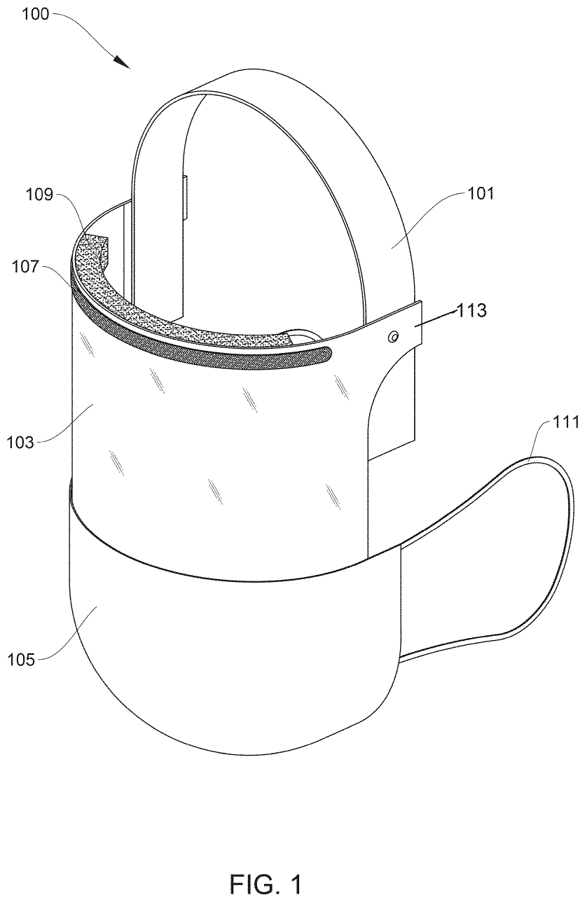

FIG. 1 shows the complete design of the Adjustable Face Shield. It is in the position where the head of a wearer gets with a multi-adjustment face shield secured by a band (101). The face shield has an upper transparent member (103) and a flexible support member made of bamboo memory foam (109) and having relief cuts. The purpose of the memory foam (109) is to provide a cushion barrier between the wearer's forehead and the transparent member. The memory foam (109) is also known as viscoelastic polyurethane foam, or low-resilience polyurethane foam (LRPu).

The face shield has a removable bottom transparent member (105) which can be attached to the upper transparent member (103) and held in place using double-sided adhesive, as illustrated by (107). (113) illustrates the closed configuration with slit fasteners to the headband (101). On the upper transparent member (103), the double-sided adhesive (107) may also serve as extended branding allowing the user the option to add ornamental crowning or branding components. The elastic bands (111) on the bottom transparent member (105) maintain tightness. Different shapes and dimensions of different faces (as well as the shapes of faces that are not well-defined, such as children and teens) do not allow a definition of universal parameters concerning the strength of the material that the elastic bands are made of. Consequently, the efficacy of the protective device is usually linked to the ability of the wearer to mold the shield to one's own face.

The memory foam (109) support member may be of many materials and configurations, as is already known in the art. The transparent member (103) may be formed of a variety of flexible materials known in the art, such for example, polyethylene terephthalate (PET), with a thickness range of 0.20 to 0.24 mm.

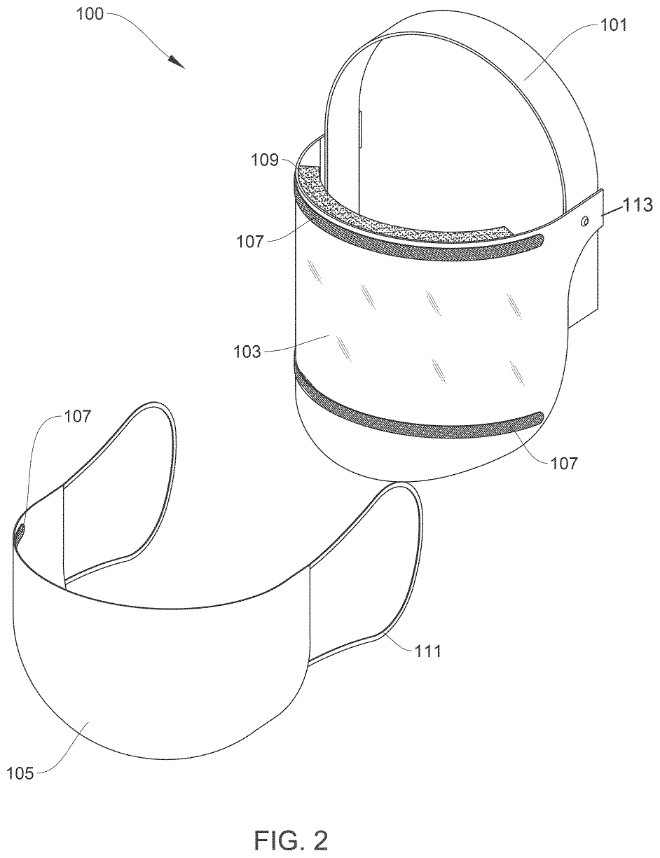

FIG. 2 shows the adjustable face shield from the anterior perspective separated from the bottom shield (105) and the band (111) of the bottom shield (105). (113) illustrates the closed configuration with slit fasteners to the headband (101)

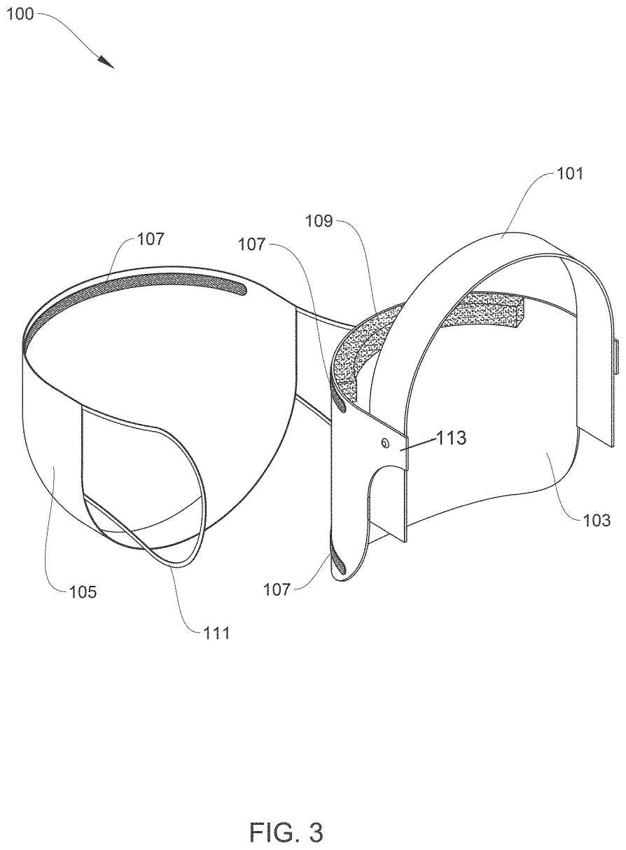

FIG. 3 shows the adjustable face shield from the posterior perspective separated from the bottom shield (105). Posterior portion of the memory foams (109) of the top shield (103) and of the bottom shield (105), and the band (101) are visible. (107) on the bottom shield attaches to the lower (107) on the top shield. (113) illustrates the closed configuration with slit fasteners to the headband (101)

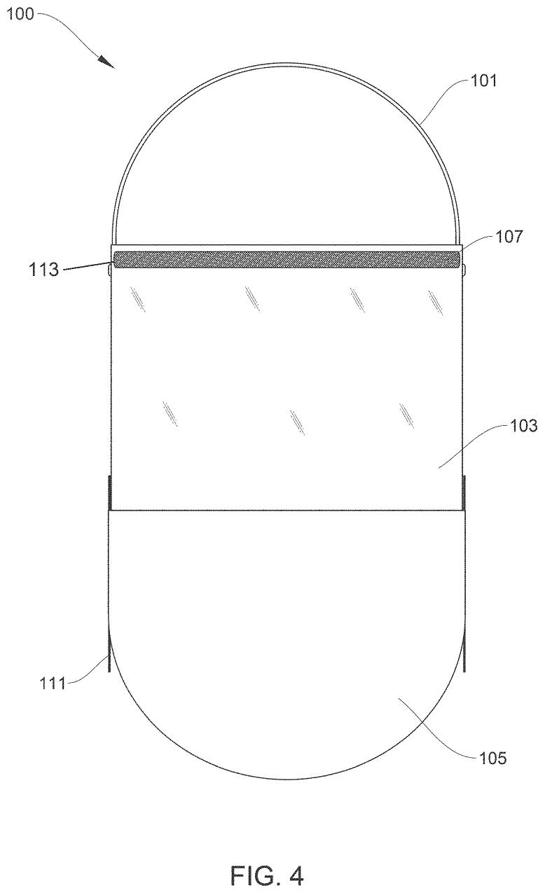

FIG. 4 shows the adjustable face shield from the anterior perspective with its complete design. Anterior portion of the double-sided adhesive (107) of the top shield (103) and of the bottom shield (105), and the band (101) are visible. (113) illustrates the closed configuration with slit fasteners to the headband (101)

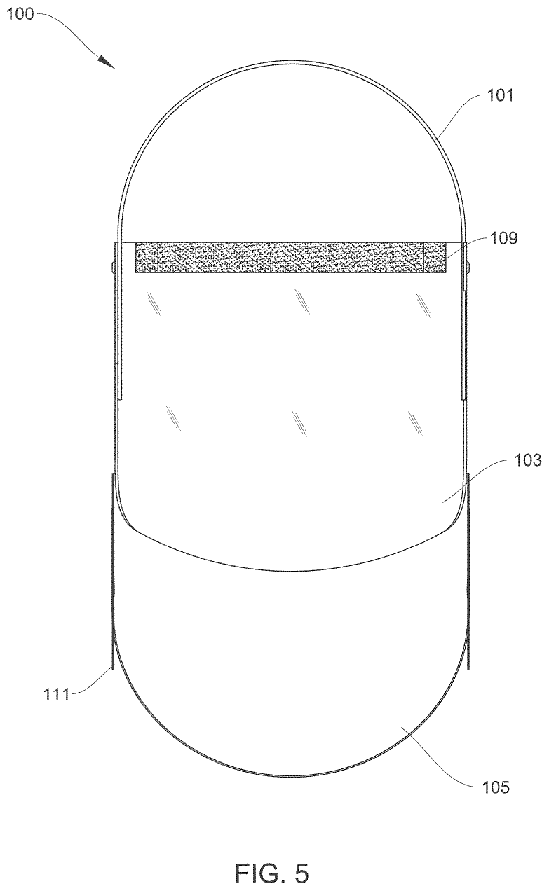

FIG. 5 shows the adjustable face shield from the posterior perspective with its complete design. Fronto-posterior portion of the memory foams (109) of the top shield (103) and of the bottom shield (105), and the band (101) are visible. (113) illustrates the closed configuration with slit fasteners to the headband (101)

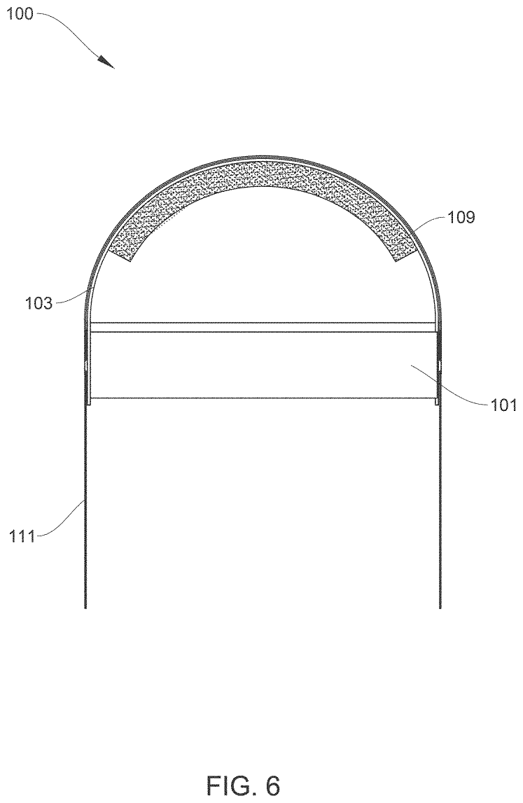

FIG. 6 shows the adjustable face shield from the dorsal perspective, seen from above. Dorsal view of the memory foams (109) of the top shield (103) and of the bottom shield (105), and the band (101) are visible. It also shows the band (111) of the bottom shield. (113) illustrates the closed configuration with slit fasteners to the headband (101)

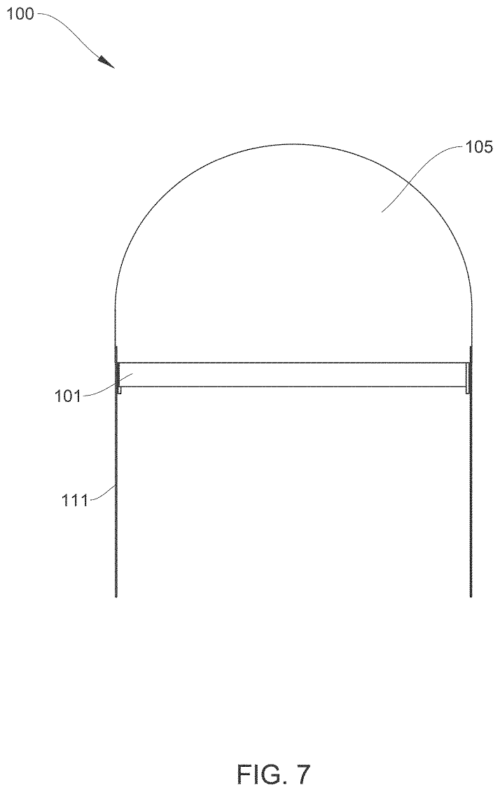

FIG. 7 shows the adjustable face shield from the ventral perspective, seen from below.

Ventral view of the bottom shield (105), and the band (101) are visible. It also shows the band (111) of the bottom shield. (113) illustrates the closed configuration with slit fasteners to the headband (101)

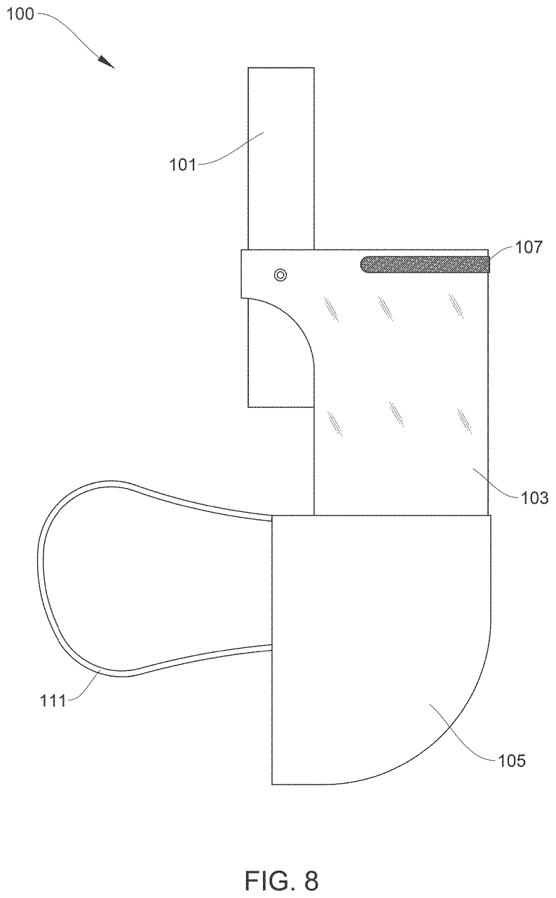

FIG. 8 shows the adjustable face shield from the lateral perspective, seen from right. Right-side view of the double-sided adhesive (107) of the top shield (103) and of the bottom shield (105), and the band (101) are visible. It also shows the band (111) of the bottom shield. (113) illustrates the closed configuration with slit fasteners to the headband (101)

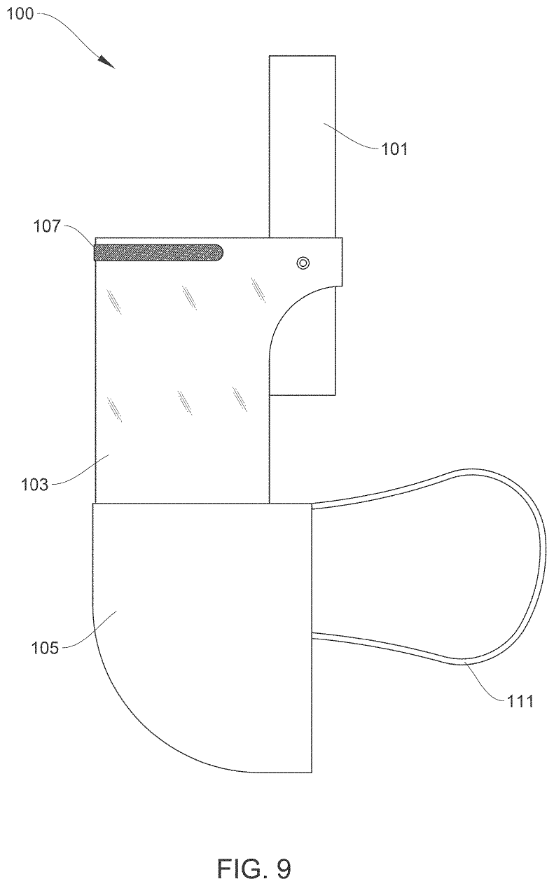

FIG. 9 shows the adjustable face shield from the lateral perspective, seen from left. Left-side view of the memory foams (109) of the top shield (103) and of the bottom shield (105), and the band (101) are visible. It also shows the band (111) of the bottom shield. (113) illustrates the closed configuration with slit fasteners to the headband (101)

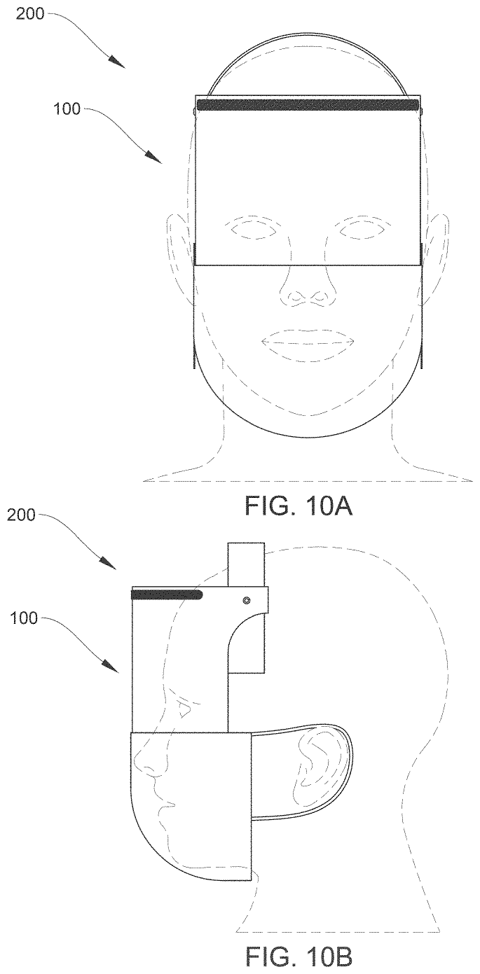

FIG. 10A shows the adjustable face shield being worn by a wearer. FIG. 10B shows the same view now seen from left.

Further considerations:

The bottom removable transparent member offers optional utility for the user. This closed configuration may be preferred as being more comfortable by some users, and may offer more substantial protection to the user's mouth and chin area, which may be desirable in certain situations.

The following additional explanations are provided for elements 101, 103, 105, 107, 109, 111, and 113, discussed above.

101: Headband. Preferably has a U-shaped band constructed of a flexible material, such as plastic. The U-shaped band is dimensioned to fit over the head of a user so that the sides of the U-shaped band extend along the sides and functionally, but removably, secure the top transparent member to the headband to the user's head. The transparent member is secured to the U-shaped band with the slit fasteners.

103: Transparent face shield. Transparent face portion is preferably made of high quality, anti-fog plastic, of a size to generally extend from the user's forehead to just below the user's nose. Types of plastic materials useful for the face portion that are optically clear, thin gauge plastics such as PVC, PET, PETG, OPS, Polycarbonate and APET. These are all rolled or sheet plastics that can be used for making face shields. The face portion is designed so that when it is in place on the user's face, an optical area in front of the user's eyes is generally flat, thus creating less optical distortion to the user.

105: Bottom transparent shield. A generally cup-shaped mask body comprising an interior surface, an opening having a peripheral edge shaped to generally follow a contour of a human face extending around at least the nasal and oral areas, a recess shaped to receive oral and nasal features of a human face, and an exterior surface opposite the interior surface. The peripheral edge has a plurality of fenestrations formed to connect both internally to the upper transparent mask via the double-sided adhesive and external sides of the mask body via the elastic bands.

107: double-sided adhesive. Rectangular sheet of flexible material with one or more strips of a first adhesive material secured to one surface parallel to a pair of edges, and one or more strips of a second adhesive material secured to the other surface juxtaposed with respect to the strips of first adhesive material. In one preferred embodiment, the first and second adhesive materials are hook and loop fastener tape, such as VELCRO®, and can be engaged with each other by pressure. A handle is provided, and pieces of the second adhesive material are attached to ornaments or branding, implements, or other items.

109: Memory foam. Silicone sealants such as various foams such as urethane foams and liquid-curable sealants are widely used for the purpose of fluid sealing, and impact insulation of industrial equipment. In order for these materials to exhibit sufficient performance of comfort, it is necessary to fill parts of an apparatus without gaps. The conventional foam member fills the gap between the forehead and top transparent member by attaching it to a portion that needs to be insulated in a compressed state, and restores the thickness by the elastic force of the foaming material itself. However, since the conventional foam member restores its original shape as soon as the pressure is released, it is necessary to attach the foam member or the assembly using the memory foam member.

111: Bands for bottom transparent member. The lower transparent mask has an earring loop attachment, the band of which extends around the back of the wearer's ear from the side of the mask loop. There is an advantage associated with providing a mask that attaches over the wearer's ear rather than winding around the back of the head. This mask is easy to wear and remove. In addition, a band extending around the back of the wearer's head may be less attractive to many wearers because the band can easily entangle the wearer's head or ruin the wearer's hairstyle. In addition to the masks held by the ear loops, masks are also known which have anti-drop bands. When not wearing a mask over the nose and mouth, the drop-proof band keeps the mask around the wearer's neck. In this way, the mask is supported on the wearer's chest and does not need to be stored separately. This is provided for quickly repositioning the mask over the wearer's nose and mouth. The anti-fall feature also frees the wearer's hands for other tasks. If wearing or removing the mask is inconvenient or not easy to use and access when not wearing, the wearer will not be able to wear the mask, which is harmful to health.

113: Slit of band, fastener. Restrains the band end from being pulled out from the slit between the first resin member and the second resin member by pressing the retaining teeth by the urging of the resin spring, and resists the urging of the resin spring.

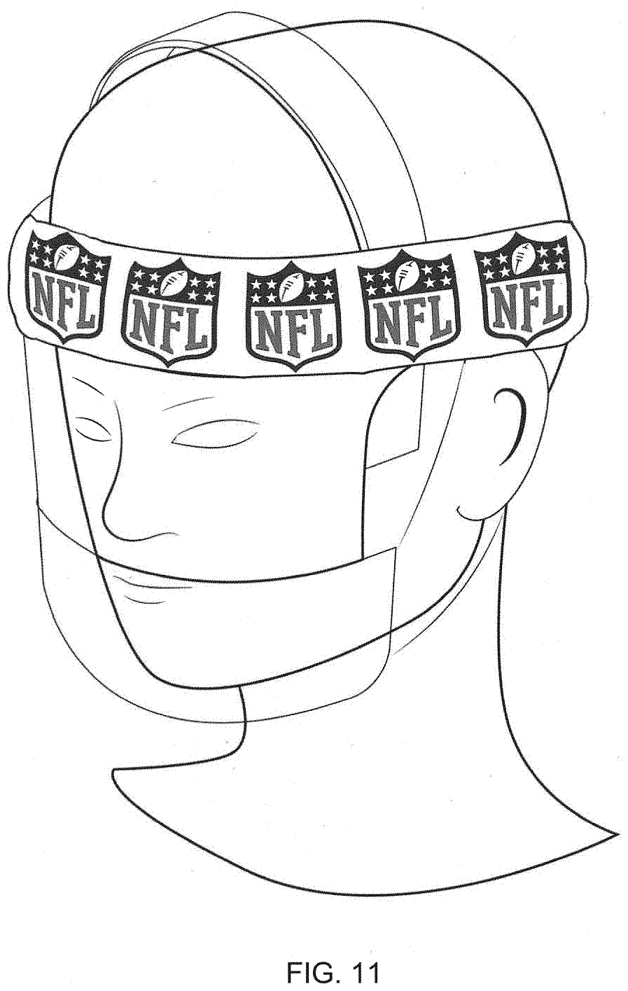

FIGS. 11-14C shows various views of the adjustable face shield with an ornamental component attached thereto via double-sided adhesive, such as hook and loop fastener tape (e.g., VELCRO (not shown), which is attached behind the ornamental front-facing band of the crowning component.

FIG. 11 is a sports team-branded component, such as an NFL team logo.

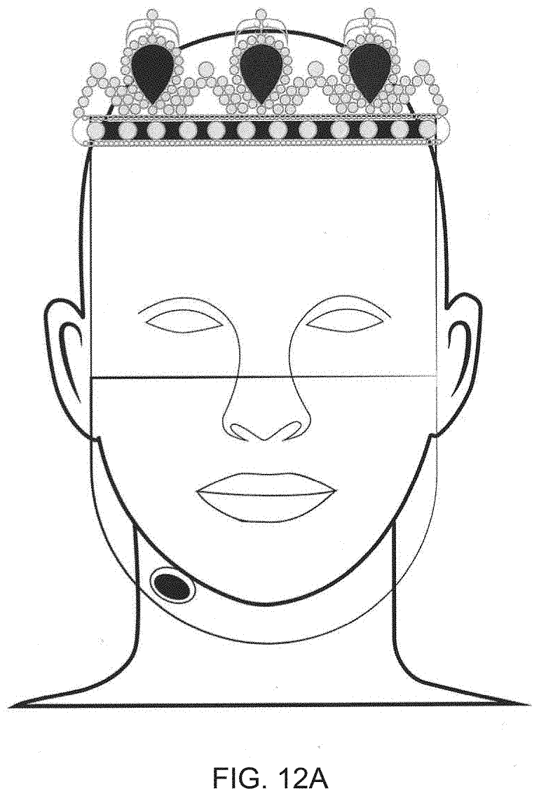

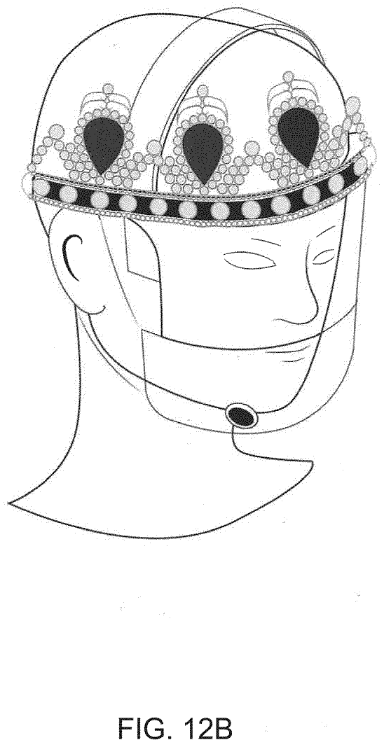

FIGS. 12A and 12B show a first embodiment of a crowning component.

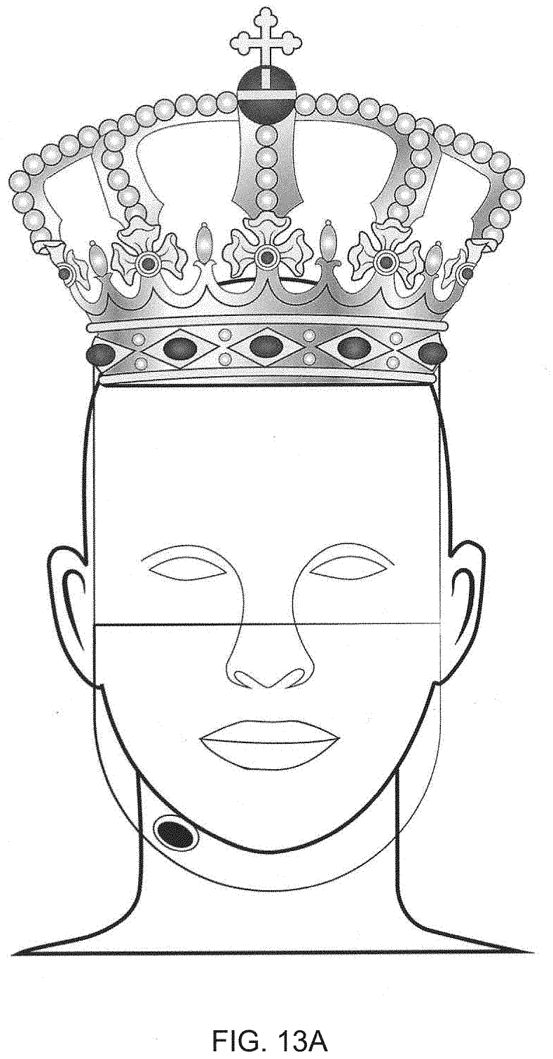

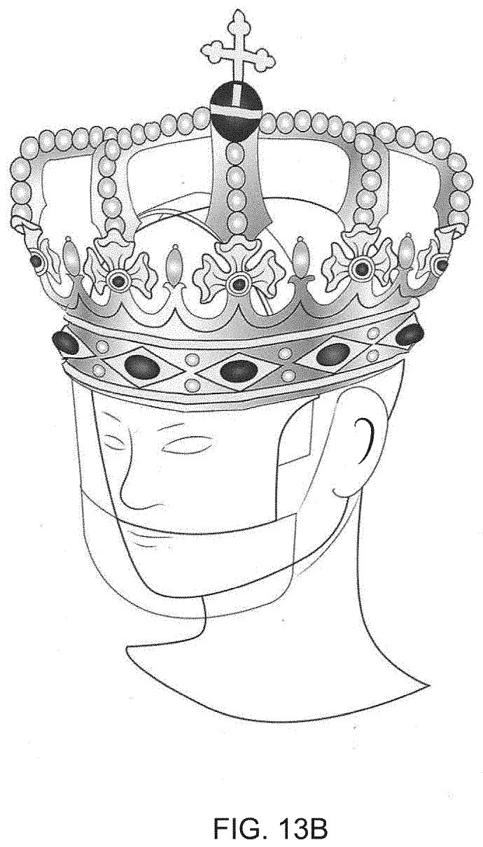

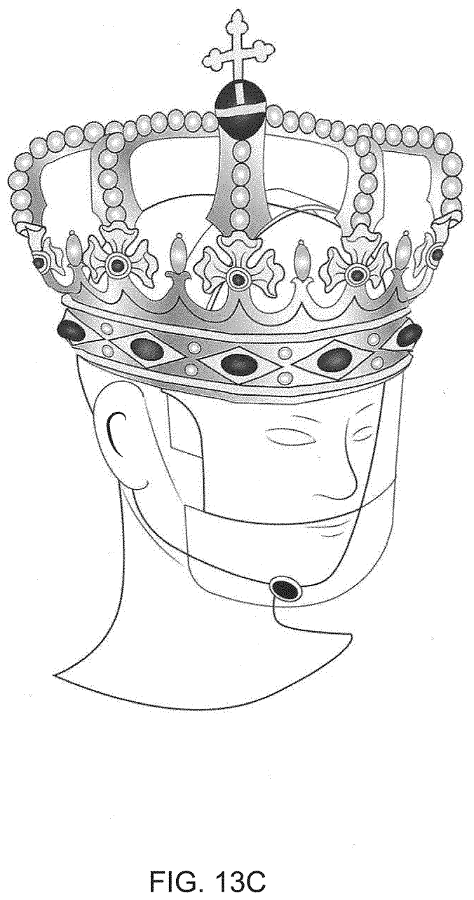

FIGS. 13A-13C show a second embodiment of a crowning component.

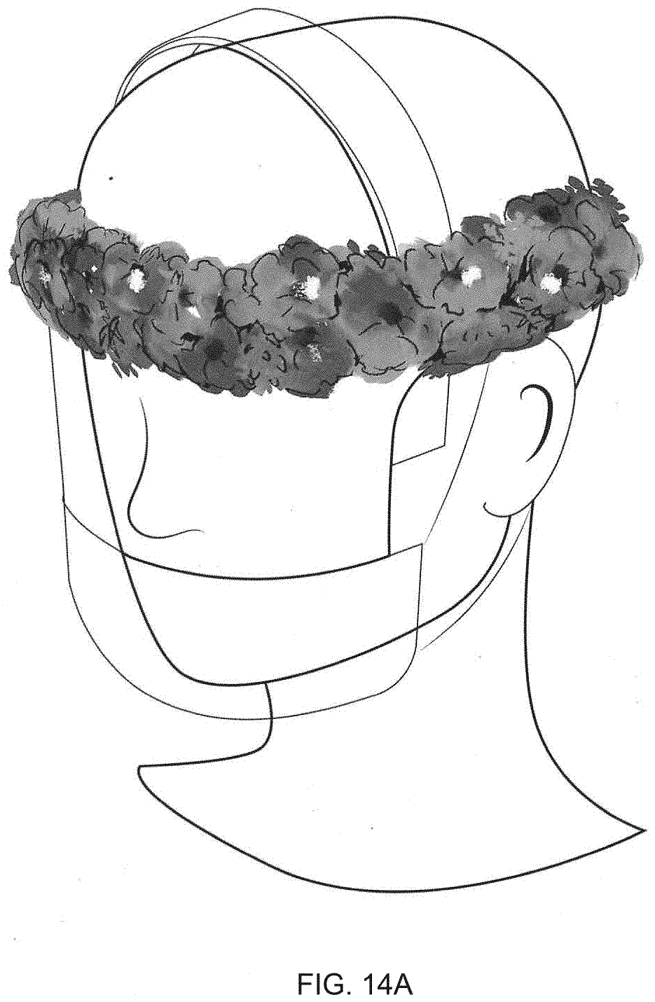

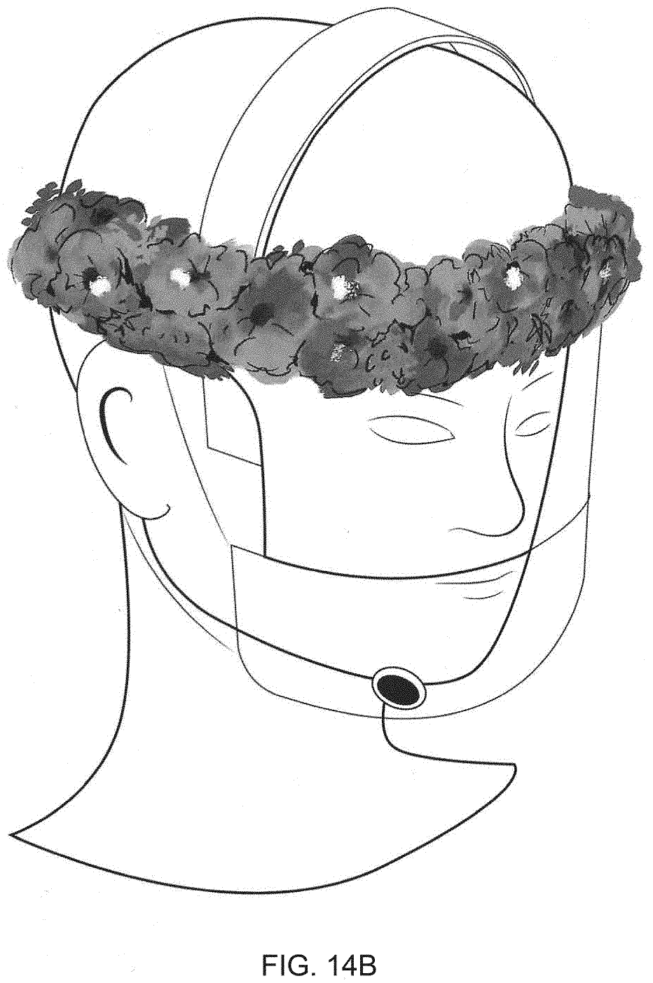

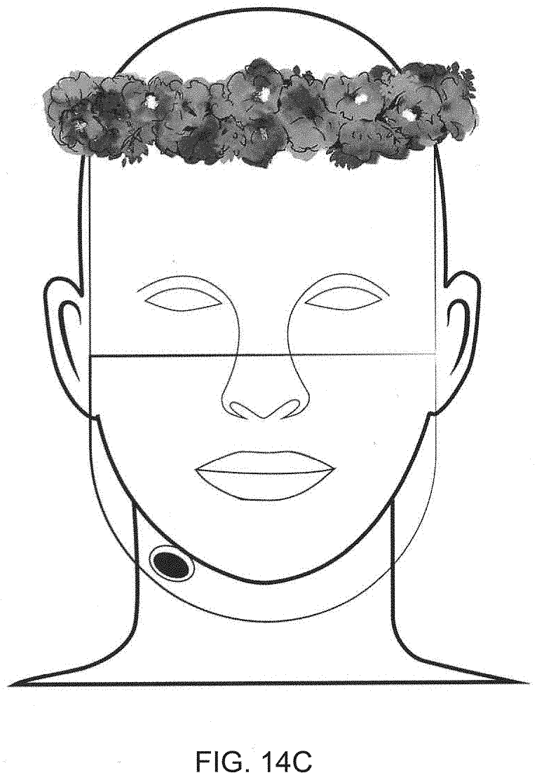

FIGS. 14A-14C show a flower crown component.

The present disclosure, therefore, is robust and not dependent upon a consistent band path. While the present disclosure has been illustrated by the description of the embodiments thereof, and while the embodiments have been described in considerable detail, it is not the intention of the applicant to restrict or in any way limit the scope of the appended claims to such detail. Additional advantages and modifications will readily appear to those skilled in the art. Therefore, the present disclosure in its broader aspects is not limited to the specific details of the representative apparatus and method, and illustrative examples shown and described. Accordingly, departures may be made from such details without departure from the spirit or scope of the applicant's general inventive concept.

It will be appreciated by those skilled in the art that various modifications and alterations could be made to the disclosure above without departing from the broad inventive concepts thereof. Some of these have been discussed above and others will be apparent to those skilled in the art. It is understood, therefore, that this disclosure is not limited to the particular embodiments disclosed, but it is intended to cover modifications within the spirit and scope of the present disclosure, as set forth in the appended claims.

I claim:

1. A face shield comprising:
a flexible, upper transparent member having a flexible headband extending from one side of the upper transparent member to an opposing side of the upper transparent member and dimensioned to extend around a portion of a head of a user, the flexible headband being configured to removably secure the upper transparent member to the head of the user;
a flexible cushion member disposed on a posterior side of the upper transparent member, the flexible cushion member being configured to provide a cushion barrier between the upper transparent member and a forehead of the user; and
a flexible, bottom member removably attached to the upper transparent member, a first side of the bottom member having a first elastic band attached thereto and a second side of the bottom member having a second elastic band attached thereto, each elastic band having both ends thereof attached to the respective side of the bottom member, whereby the first and second elastic bands are each configured to loop around a respective ear of the user to removably secure the bottom member to the head of the user.

2. The face shield of claim 1, further comprising an adhesive strip positioned along an anterior side of the upper transparent member, the adhesive strip being configured to removably receive a decorative component thereon.

3. The face shield of claim 2, wherein the adhesive strip is a double-sided adhesive strip.

4. The face shield of claim 1, wherein the flexible cushion member is constructed of memory foam.

5. The face shield of claim 4, wherein the flexible cushion member is constructed of bamboo memory foam.

6. The face shield of claim 1, wherein the upper transparent member defines a thickness between approximately 0.20 mm and approximately 0.24 mm.

7. The face shield of claim 1, wherein at least one end of the headband is removably attached to the upper transparent member.

8. The face shield of claim 1, wherein the bottom member is also a transparent member.

9. The face shield of claim 1, wherein the upper transparent member and the bottom member are removably attached via hook-and-loop fastener strips.

10. The face shield of claim 1, wherein a posterior side of the bottom member is removably attached to an anterior side of the upper transparent member.

11. A face shield comprising:
a flexible, upper transparent member having a flexible headband extending from one side of the upper transparent member to an opposing side of the upper transparent member and dimensioned to extend around a portion of a head of a user, the flexible headband being configured to removably secure the upper transparent member to the head of the user, wherein at least one end of the headband is removably attached to the upper transparent member;
a memory foam, flexible cushion member disposed on a posterior side of the upper transparent member, the flexible cushion member being configured to provide a cushion barrier between the upper transparent member and a forehead of the user;
an adhesive strip positioned along an anterior side of the upper transparent member, the adhesive strip being configured to removably receive a decorative component thereon; and
a flexible, bottom transparent member removably attached to the upper transparent member, a first side of the bottom transparent member having a first elastic band attached thereto and a second side of the bottom transparent member having a second elastic band attached thereto, each elastic band having both ends thereof attached to the respective side of the bottom transparent member, whereby the first and second elastic bands are each configured to loop around a respective ear of the user to removably secure the bottom transparent member to the head of the user,
wherein a posterior side of the bottom transparent member is removably attached to the anterior side of the upper transparent member via hook-and-loop fastener strips.

12. The face shield of claim 11, wherein the adhesive strip is a double-sided adhesive strip.
